(19) United States
(12) Patent Application Publication
(10) Pub. No.: US 2007/0158457 A1
(43) Pub. Date: Jul. 12, 2007

(54) Liquid disseminating device

(76) Inventors: Colin Brown, Bracknell Berkshire (GB); Guy Edward Naish, Bicester Oxfordshire (GB); Kishen Gohil, New Malden Surrey (GB)

Correspondence Address: CURATOLO SIDOTI CO., LPA, 24500 CENTER RIDGE ROAD, SUITE 280, CLEVELAND, OH, 44145, US

(21) Appl. No.: 10/569,891

(22) PCT Filed: Aug. 20, 2004
(86) PCT No.: PCT/CH04/00527

§ 371 Date: Oct. 25, 2006

(30) Foreign Application Priority Data
Sep. 2, 2003 (GB) 0320460.9

(51) Int. Cl. A61L 9/04

(52) U.S. Cl. 239/44

(57) ABSTRACT

An apparatus adapted to disseminate volatile liquid into an atmosphere comprises a reservoir containing volatile liquid and a liquid transfer member that transfers liquid from the reservoir to the atmosphere via an orifice in the reservoir. The reservoir orifice is closed by a puncturable membrane, and the transfer member is mounted adjacent to the orifice on the side of the membrane remote from the liquid and adapted such that it can be pushed through the puncturable membrane into, and then held in contact with, the liquid in the reservoir, to permit the flow of the liquid from the reservoir to the atmosphere. The liquid transfer member preferably comprises capillary channels. The apparatus has the advantage of being leakproof in transit.

This invention relates to an apparatus for disseminating a volatile liquid into an atmosphere.

Many commercially-available apparatus for the dissemination of volatile liquids, such as fragrances and insecticides, into an atmosphere comprise a reservoir of volatile liquid and, extending therefrom, a liquid transfer member, typically a porous wick. In a more recent development, it has been proposed to replace the wick with an external capillary member, that is, a member bearing external channels of capillary dimensions. Such a member has been described in U.S. Pat. No. 4,913,350. Such members have the advantage of avoiding the undesirable fractionating effect that occurs particularly with multi-component fragrances, with one component evaporating into the atmosphere before the others.

However, apparatus utilising this technology are prone to leakage during transport, and this has been a major factor in the lack of acceptance of this otherwise superior transfer member technology.

It has now been found that this problem can be overcome by a particular design of apparatus. The invention therefore provides an apparatus adapted to disseminate volatile liquid into an atmosphere, the apparatus comprising a reservoir containing volatile liquid and a liquid transfer member that transfers liquid from the reservoir to the atmosphere via an orifice in the reservoir, the reservoir orifice being closed by a puncturable membrane, and the transfer member being mounted adjacent to the orifice on the side of the membrane remote from the liquid and adapted such that it can be pushed through the puncturable membrane into, and then held in contact with, the liquid in the reservoir, to permit the flow of the liquid from the reservoir to the atmosphere.

The reservoir can be any suitable reservoir of any suitable size and material. It has an orifice that may be sealed by a puncturable membrane, which may be any puncturable membrane known to the art, for example metal foil or plastics. The membrane may be across a neck of the reservoir, which comprises the orifice, or it may be one or more membranes in a cap that is attached over the orifice.

The liquid is transferred from the reservoir to the atmosphere by means of a transfer member. In using the word “member” in the singular, the plural is also included, that is, a plurality of such members may be used.

The transfer member may be any suitable transfer member. It may, for example, be a solid material that is inherently porous, and many such materials having the necessary porosity and rigidity are known to the art. However, it is preferably an external capillary member, that is, a member bearing on its surface a plurality of capillary channels.

The transfer member is mounted adjacent to the orifice and is adapted to be pushed through the membrane to reach the liquid in the reservoir and convey it to the atmosphere. This can be achieved by any convenient means. One means is to provide the orifice of the reservoir with a neck and the member bearing the transfer member as part of a cap that can be attached to this neck by any convenient means, for example, by screw threads or by a snap fitting. In one embodiment, the apparatus is provided as two components, a sealed reservoir and a cap, which can be combined for use. This has two advantages, namely (a) there is no possibility of accidental opening of the reservoir in transport, and (b) the reservoir may be provided as a refill, enabling the neck to be used a number of times.

There are many possible ways of providing the required transfer member that will be immediately evident to the skilled person. In a preferred embodiment, the transfer member forms part of a cap as hereinabove described, the cap being vented to atmosphere and having suitable fittings to allow attachment to the reservoir in such a position that the transfer member can puncture the membrane, when required. The transfer member itself may be located, and slidable, within the cap by any convenient means. It may puncture the membrane by any convenient means, for example by means of points or edges provided on its bottom.

The transfer member may be of substantial thickness, meaning that, when it is pushed through the membrane, most of, or even all, of the membrane is removed. This allows the possibility of leakage, and it is preferred that the transfer means puncture the membrane, leaving it essentially intact and therefore less prone to leakage. This may be achieved by using a number of relatively narrow, relatively thin transfer members arranged in any suitable way. Thus, in an especially preferred embodiment, the capillary member consists of a number (typically 2-6) of relatively narrow, relatively thin, typically planar members arranged in a circular pattern and held together at their tops by any suitable structure. At the bottom of each transfer member is a point or edge adapted to puncture the membrane. In an especially preferred embodiment, the transfer member comprises a plurality of narrow, thin members bearing external capillaries.

The apparatus is brought into operation by pushing the transfer member through the membrane and into the liquid. Liquid can then flow up the transfer member and be disseminated into the atmosphere. This can be arranged by any convenient means. For example, the transfer member may be located within a flexible outer cap, which, when pushed, will distort, while the rigid transfer member does not and is pushed into puncturing the membrane. In a further embodiment, the transfer member is provided with locking means, such as a snap fitting, that prevents its withdrawal from contact with the liquid. In a further embodiment, the outer cap is provided with longitudinal slots, which, when the cap is pressed to bring the capillary member into contact with the liquid, open to allow release of liquid into the atmosphere.

The invention additionally provides a method of providing secure transport of a volatile liquid to be released into an atmosphere, followed by efficient release of that liquid into the atmosphere, comprising the provision of a reservoir of volatile liquid having an orifice closed by a puncturable membrane, and puncturing that membrane by pushing through it at least one liquid transfer member adapted to puncture the membrane, to such an extent that the transfer member comes into liquid-transferring contact with the liquid in the reservoir and maintaining this liquid-transferring contact.

The invention is now further described with reference to the drawings, which depict preferred embodiments and which are not intended to be limiting on the scope of the invention in any way.

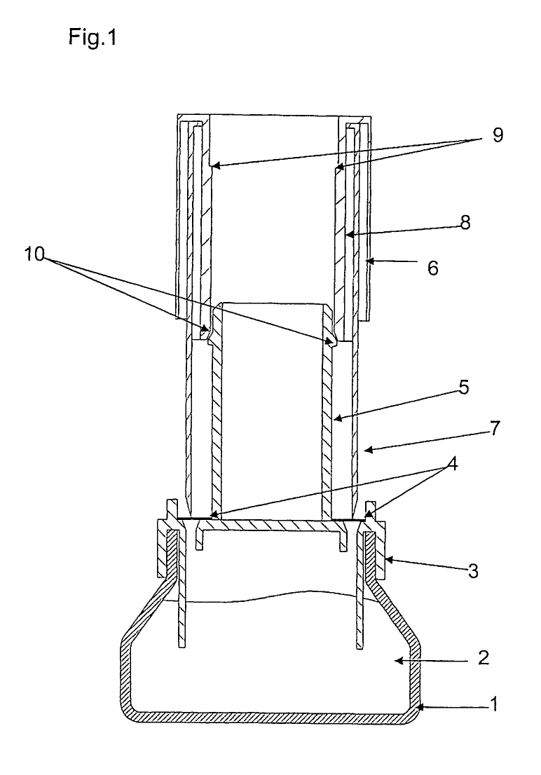

FIG. 1 is a longitudinal cross-section through an embodiment of the invention, prior to its activation.

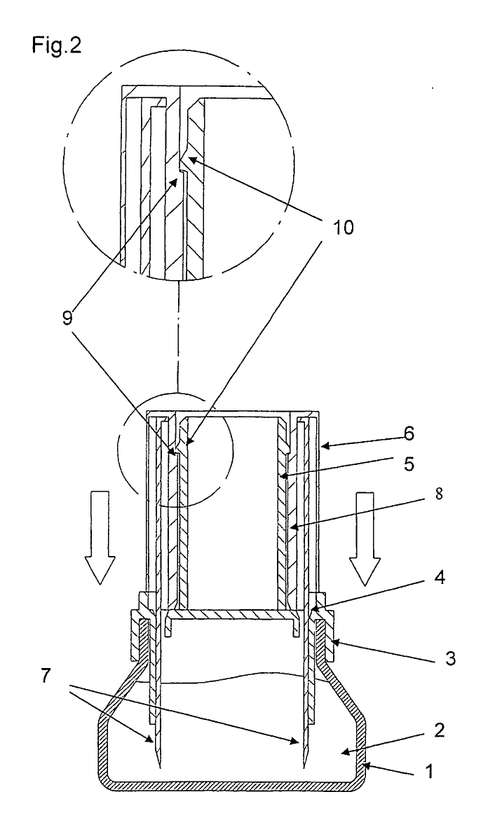

FIG. 2 is a longitudinal cross-section through the embodiment of FIG. 1, showing it in activated position, with detailed inset of a locking mechanism.

A reservoir 1 contains a volatile liquid 2 for dissemination into an atmosphere. To the open neck of this reservoir is fitted a closure 3, that contains four orifices, sealed with foil 4. The closure has an extended neck 5, on which rests a cap 6. From this cap depend four capillary members 7, these being flat members whose surface comprises capillary channels. The four capillary members correspond with the four foil-closed orifices, and the capillary members end adjacent to the foil in points capable of penetrating the foil.

The cap has an inner sleeve 8 that is of the same diameter as the inner diameter of the neck 5, enabling one to slide over the other. At the top of the sleeve are indentations 9. These are adapted to mate with matching projections 10 on the neck 5 and hold the cap in place when it is depressed. The material of the neck 5 is sufficiently flexible such that, when the cap 6 is depressed, the sleeve 8 can slide over the projections, until the indentations 9 are reached, at which point the projections spring out, locking the cap in place.

FIG. 2 shows the cap in its depressed position. The capillary members 7 are pushed through the foils 4 to the liquid 2. The projections 10 in the neck 5 are locked into the indentations 9 in the cap inner sleeve 8. Liquid can flow up the capillary members 7 for evaporation into the atmosphere.

1. An apparatus adapted to disseminate volatile liquid into an atmosphere, the apparatus comprising a reservoir containing volatile liquid and a liquid transfer member that transfers liquid from the reservoir to the atmosphere via an orifice in the reservoir, the reservoir orifice being closed by a puncturable membrane, and the transfer member being mounted adjacent to the orifice on the side of the membrane remote from the liquid and adapted such that it can be pushed through the puncturable membrane into, and then held in contact with, the liquid in the reservoir, to permit the flow of the liquid from the reservoir to the atmosphere.

2. An apparatus according to claim 1, in which the liquid transfer member comprises a plurality of relatively narrow, relatively thin transfer members.

3. An apparatus according to claim 1, in which the liquid transfer member comprises capillary channels.

4. A method of providing secure transport of a volatile liquid to be released into an atmosphere, followed by efficient release of that liquid into the atmosphere, comprising the provision of a reservoir of volatile liquid having an orifice closed by a puncturable membrane, and puncturing that membrane by pushing through it at least one liquid transfer member adapted to puncture the membrane, to such an extent that the transfer member comes into liquid-transferring contact with the liquid in the reservoir and maintaining this liquid-transferring contact.

5. A method according to claim 4, in which the reservoir and the transfer member are provided in separate components that are combined when dissemination of the liquid is desired.

6. The apparatus according to claim 2, in which the liquid transfer member comprises capillary channels.

7. The apparatus according to claim 1, in which the reservoir orifice is not below the reservoir during operation.

8. The apparatus according to claim 7, in which the liquid transfer member comprises a plurality of relatively narrow, relatively thin transfer members.

9. The apparatus according to claim 8, in which the liquid transfer member comprises capillary channels.

10. The apparatus according to claim 9, in which the liquid transfer from the reservoir is due primarily to capillary action.
